(19) United States
(12) Patent Application Publication
(10) Pub. No.: US 2020/0263121 A1
(43) Pub. Date: Aug. 20, 2020

(54) METHOD FOR COUNTING NUMBER OF MICROORGANISMS

(71) Applicant: JNC CORPORATION, Tokyo (JP)

(72) Inventors: Hajime TERAMURA, Kanagawa (JP); Aoi FUJIWARA, Kanagawa (JP)

(73) Assignee: JNC CORPORATION, Tokyo (JP)

(21) Appl. No.: 16/651,357

(22) PCT Filed: Jun. 4, 2018
(86) PCT No.: PCT/JP2018/021321

§ 371 Date: Mar. 27, 2020

(30) Foreign Application Priority Data
Sep. 29, 2017 (JP) 2017-190691

(51) Int. Cl. C12M 1/34; C12M 1/00; C12Q 1/06; C12N 1/20

(52) U.S. Cl. CPC C12M 41/36; C12M 23/14; C12N 1/20; C12M 23/22; C12Q 1/06; C12M 23/20

(57) ABSTRACT

Provided is a method for simply and accurately counting the number of microorganisms in an analyte containing carbonate ion or bicarbonate ion in a dialysis fluid or the like. A counting method for the number of microorganisms includes: a step of adding the analyte to a composition for preparing a medium for counting the number of microorganisms, and containing (a) polyacrylic acid and/or a salt thereof, (b) hydroxide of alkaline earth metal and (c) a nutritional ingredient, and mixing the resulting mixture; a step of culturing microorganisms contained in the analyte; and a step of counting the number of colonies of the microorganisms.

### TECHNICAL FIELD

The invention relates to a method for simply counting the number of microorganisms in an analyte, and particularly to a method that can be applied to an analyte containing carbonate ion or bicarbonate ion in dialysis water or the like.

### BACKGROUND ART

In drinking water, soft drink, industrial water, pharmaceutical water, dialysis water or the like, a degree of contamination by microorganisms is required to be small, and the number of existing microorganisms is ordinarily known to be significantly small. In particular, criteria are specified to be 100 CFU/mL or less in viable counts for drinking water, and 10 CFU/mL or less in viable counts for water for dialysis, and monitoring for understanding the accurate number of microorganisms on a regular basis in controlling the criteria described above is important (Non-patent literature No. 1).

In detection of the microorganisms in food or the like, a method is ordinarily applied in which a small amount of an analyte suspension or the like in an amount of about 1 milliliter is subjected to pour plate culture. However, for the analyte such as drinking water or the like in which an amount of existing microorganisms is significantly small, the number of microorganisms is unable to be accurately counted in several cases according to the method described above. For example, when 10 CFU or less of bacteria exists in 100 milliliters of the analyte, if only 1 milliliter thereof is provided therefor as the analyte, the microorganisms are unable to be detected in several cases, and a contamination state is unable be accurately understood.

In order to simply and accurately count the number of microorganisms in a liquid analyte, particularly the number of a small amount of microorganisms existing in a large volume of liquid analyte as described above, the present inventors have so far proposed a method by a medium using a gelling agent such as sodium polyacrylate (application for patent JP 2016-189545). More specifically, the method includes a method in which operation is simplified by directly adding a fluid analyte as a solvent composing a medium into a gelling agent to emerge colonies by culturing microorganisms in an analyte in the medium, and to visualize the microorganisms (Reference Example 1 described later).

### CITATION LIST

### Non Patent Literature

NPL 1: The Japanese Pharmacopoeia, 17th Edition, G8 Water in General Information
NPL 2: Preparation of powdery dialysis fluid for artificial kidney, Attachment LYMPACK TA3 (Nipro Corporation)

### SUMMARY OF INVENTION

### Technical Problem

However, when the method described above is applied to a case where a dialysis fluid is applied as an analyte, a medium becomes cloudy and a new problem of reduction of visibility of colonies of microorganisms has occurred. The present inventors have found that sodium carbonate or sodium bicarbonate ordinarily contained in a large amount in the dialysis fluid (Non-patent literature No. 2) reacts with sodium polyacrylate to generate a carbon dioxide gas to generate uncountable bubbles in the medium.

In such a situation, an object of the invention is to provide a method in which of simply and accurately counting a small amount of microorganism counts existing in an analyte containing carbonate ion or bicarbonate ion in the dialysis fluid or the like is simply and accurately counted by suppressing generation of a carbon dioxide gas derived from the dialysis fluid.

### Solution to Problem

The present inventors have diligently continued to conduct study in order to solve the problems as described above. As a result, the present inventors have found that, if hydroxide of alkaline earth metal, such as calcium hydroxide, is added as a component of a medium containing sodium polyacrylate, generation of a carbon dioxide gas can be suppressed and visibility upon counting the number of microorganisms can be secured, and have completed the present invention.

More specifically, the invention includes the items described below.

Item 1. Use of a composition for counting the number of microorganisms, wherein the composition comprises (a) polyacrylic acid and/or a salt thereof, (b) hydroxide of alkaline earth metal and (c) a nutritional ingredient.

Item 2. The Use of item 1, wherein the composition further comprises (d) a coloration reagent.

Item 3. The Use of item 1 or 2, wherein (b) the hydroxide of alkaline earth metal is selected from the group of calcium hydroxide, magnesium hydroxide, barium hydroxide, strontium hydroxide, radium hydroxide and beryllium hydroxide.

Item 4. Use of culture equipment for counting the number of microorganisms, wherein the culture equipment comprises (a) polyacrylic acid and/or a salt thereof, (b) hydroxide of alkaline earth metal, (c) a nutritional ingredient, and a culture vessel.

Item 5. A composition for preparing a medium for counting the number of microorganisms, containing (a) polyacrylic acid and/or a salt thereof, (b) hydroxide of alkaline earth metal and (c) a nutritional ingredient.

Item 6. The composition according to item 5, further containing (d) a coloration reagent.

Item 7. The composition according to item 5 or 6, wherein (b) the hydroxide of alkaline earth metal is selected from the group of calcium hydroxide, magnesium hydroxide, barium hydroxide, strontium hydroxide, radium hydroxide and beryllium hydroxide.

Item 8. Culture equipment for counting the number of microorganisms, including the composition according to any one of items 5 to 7, and a culture vessel.

Item 9. A counting method for the number of microorganisms, including a step of adding an analyte to the composition according to any one of items 5 to 7 and mixing the resulting mixture, a step of culturing microorganisms contained in the analyte, and a step of counting the number of colonies of the microorganisms.

Item 10. The counting method according to item 9, wherein the analyte contains 0.005 mol/100 mL or more of carbonate ion or bicarbonate ion.

Item 11. The counting method according to item 9 or 10, wherein the number of microorganisms in the analyte is 0.1 CFU/mL or less.

Item 12. The counting method according to any one of items 9 to 11, wherein a weight of the analyte is 10 to 10,000 times a weight of (a) the polyacrylic acid and/or the salt thereof in the composition.

### Advantageous Effects of Invention

According to the invention, generation of a carbon dioxide gas can be suppressed even for an analyte containing carbonate ion or bicarbonate ion, and the number of microorganisms in the analyte can be simply and accurately counted. In particular, quantitative detection can be achieved even for a small amount of microorganism counts existing in a large amount of analyte.

### BRIEF DESCRIPTION OF DRAWINGS

FIG. 1 is a photograph of red colonies in 100 milliliters of a gel-form medium in Reference Example 1.

FIG. 2 is a photography of 100 milliliters of a gel-form medium in Comparative Example 1.

FIG. 3 is a photography of 100 milliliters of a gel-form medium in Example 1.

### DESCRIPTION OF EMBODIMENTS

A composition of the invention essentially contains (a) polyacrylic acid and/or a salt thereof, (b) hydroxide of alkaline earth metal, and (c) a nutritional ingredient.

The composition of the invention is a material for preparing a medium for counting the number of microorganisms. The preparation is ordinarily performed by adding a liquid analyte containing the microorganisms to be counted, and mixing the analyte into the composition, as a solvent of gel directly composing the medium. In such a use aspect, the composition of the invention and the medium prepared using the composition are different from an existing medium for microorganisms.

Then, (a) polyacrylic acid and/or the salt thereof play a role of a gelling agent composing the medium for culturing and counting target microorganisms. Polyacrylic acid and/or the salt thereof form uniform gel by being mixed with the liquid analyte. Such gel formation ordinarily needs dissolution by heating or cooling, and therefore operation is simple, and growth of the target microorganisms is not hindered.

The gel formed has ordinarily no flowability, and therefore the number of existing microorganisms can be accurately counted. Moreover, the gel ordinarily causes no syneresis, and therefore the number of colonies of existing microorganisms can be accurately counted.

Moreover, the gel formed of polyacrylic acid and/or the salt thereof is ordinarily transparent. Thus, the colonies of the microorganisms can be accurately detected by visual observation.

The gelling agent such as agar, carragheenan and locust bean gum is generally used for the medium for microorganisms, or the like, but the agents described above require heating upon solidifying a liquid solvent, and therefore are unsuitable for directly solidifying the liquid analyte containing the microorganisms. Moreover, the gel prepared by solidification using the gelling agent described above is also unsuitable therefor in view of low transparency.

Moreover, polyvinyl alcohol is hard to be homogeneously mixed with the liquid solvent, and also has a problem of easily causing syneresis. Moreover, xanthane gum is also hard to be homogeneously mixed with the liquid solvent to easily form lumps, in which gel solidified also easily becomes opaque.

Carboxymethyl cellulose is unable to solidify the liquid analyte to form flowable gel, and therefore is unsuitable for quantitative detection of the microorganisms.

When sodium polyacrylate is used as polyacrylic acid and/or the salt thereof, from a viewpoint of solidifying capability, a material having a degree of polymerization of 10,000 or more is preferred, and a material having a degree of polymerization of 22,000 or more is further preferred. Moreover, the material may be crosslinked or need not be crosslinked.

A concentration of sodium polyacrylate when in use according to the invention is not particularly limited, but is preferably 0.01 g/100 mL to 10 g/100 mL, and further preferably 0.5 g/100 mL to 5 g/100 mL.

Moreover, when (a) any other polyacrylic acid and/or a salt thereof is used, the concentration when in use only needs to be in the range in which solidified gel is formed, as long as advantageous effects of the invention are not adversely affected.

Then, (b) the hydroxide of alkaline earth metal is a material for suppressing generation of a carbon dioxide gas by reaction of the polyacrylic acid or the like with carbonate ion or bicarbonate ion in the analyte.

The hydroxide of alkaline earth metal is not particularly limited, and specific examples thereof preferably include calcium hydroxide, magnesium hydroxide, barium hydroxide, strontium hydroxide, radium hydroxide and beryllium hydroxide, and calcium hydroxide is particularly preferred in view of handling and an influence on the growth of microorganisms.

In addition, even when the materials described above are added thereto, transparency of the gel formed of polyacrylic acid or the like and gelation are not hindered.

A concentration of (b) the hydroxide of alkaline earth metal when in use according to the invention is not particularly limited, but is preferably a concentration equal to or less than the mole number of carbonate ion and bicarbonate ion contained in the analyte. For example, when a general dialysis fluid is applied as the analyte, and calcium hydroxide is used as (b) the hydroxide of alkaline earth metal, a concentration thereof when in use is preferably 0.01 g/100 mL to 10 g/100 mL, and further preferably 0.1 g/100 mL to 0.5 g/100 mL.

Then, (c) the nutritional ingredient is applied for growing the target microorganisms.

The nutritional ingredient is not particularly limited, and specific examples thereof preferably include peptone, an animal meat extract, a yeast extract and a fish meat extract.

As described in Non-patent literature No. 1, in testing of drinking water, use of a standard agar medium is recommended, and in testing of pharmaceutical water or dialysis water, use of an R2A agar medium is recommended. Therefore, a bouillon medium in which agar is excluded from the agar media described above, or an ingredient equivalent thereto is preferably applied as a component of the composition according to the invention.

Then, the composition of the invention further preferably contains (d) the coloration reagent. The reason is that the colonies of the microorganisms, the colonies being produced by culture, are easily detected and counted as colored colonies.

Specific examples of the coloration reagent include a redox indicator including 2,3,5-triphenyltetrazolium chloride (TTC) and tetrazolium violet. The indicator described above can be preferably used when all kinds of microorganisms existing in the analyte are desirably counted. When TTC is used, a concentration when in use is preferably 1 mg/L to 100 mg/L, and further preferably 10 mg/L to 50 mg/L.

Moreover, as the coloration reagent, such a material may be used as a substrate (hereinafter, referred to as “enzyme substrate”) to an enzyme owned only by a specific microorganism species, and a compound that can release a pigment compound by being decomposed. The material described above can be preferably used when the specific microorganisms are desirably counted.

Here, the pigment compound may be any of a compound colored under visible light and a fluorescent-colored compound. Specific examples of a functional group that can be released as the colored compound under visible light include a 5-bromo-4-chloro-3-indoxyl group, and 5-bromo-4-chloro-3-indole released is oxidized and fused into 5,5′-dibromo-4,4′-dichloro-indigo, and colored blue. Specific examples of a functional group that can be released as the fluorescent-colored compound include a 4-methyl umbelliferryl group, and 4-methylumbelliferone released emits fluorescence under irradiation with ultraviolet light.

To take the enzyme substrate as an example, when the target microorganisms are a Coliform group, 5-bromo-4-chloro-3-indoxlyl-beta-D-galactopyranoside (X-GAL) or 5-bromo-4-chloro-3-indoxyl-beta-D-glucuronic acid can be preferably used, in a case of Staphylococcus aurei, 5-bromo-4-chloro-3-indoxyl phosphate (X-phos) can be preferably used, in a case of Enterococcus, 5-bromo-4-chloro-3-indoxlyl-beta-D-glucopyranoside (X-GLUC) can be preferably used, and in a case of fungi, X-phos, 5-bromo-4-chloro-3-indoxyl acetate or 5-bromo-4-chloro-3-indoxyl butyrate can be preferably used, respectively. Further, when all of the microorganism species are desirably detected, all of the substances described above may be combined and used.

A concentration of the enzyme substrate when in use is preferably 0.01 g/L to 1.0 g/L, and further preferably 0.2 g/L to 1.0 g/L.

The composition of the invention may further contain a selective substance, an antibacterial substance, inorganic salts, saccharides, a thickening agent, a pH adjuster or the like, as long as the ingredient does not adversely affect advantageous effects of the invention.

Specific examples of the selective substance include an antibiotic such as polymixin B and vancomycin, and a surfactant such as sodium lauryl sulfate (SDS), Tween 80 and a bile salt such as sodium cholate.

Specific examples of the antibacterial substance include polylysine, protamine sulfate, glycine and sorbic acid.

Specific examples of the inorganic salts include an inorganic acid metal salt such as sodium chloride and sodium thiosulfate, and an organic acid metal salt such as sodium pyruvate, ferric ammonium citrate and sodium citrate.

Specific examples of the saccharides include glucose, lactose, sucrose, xylose, cellobiose and maltose.

Specific examples of the viscosity improver include starch and a derivative thereof, hyaluronic acid, an acrylic acid derivative, polyether and collagen.

Specific examples of the pH adjuster include sodium carbonate, sodium hydrogencarbonate and citric acid. In addition, from a viewpoint of the growth of the target microorganisms, the composition according to the invention is such a composition to be preferably 6.0 to 8.0, and further preferably 6.5 to 7.5 in the pH when in use.

The composition of the invention may be provided as culture equipment for counting the number of microorganisms in combination with a culture vessel.

Such a culture vessel is vessel for directly housing the liquid analyte therein ordinarily without applying treatment such as concentration and dilution thereto, and mixing the liquid analyte with the composition according to the invention therein to cause gelation of the polymer compound contained in the composition to form a medium, and culturing the microorganisms.

A form of the culture vessel is not particularly limited, and may be a vessel as long as a required amount of the fluid analyte can be sufficiently housed. For example, in order to mix the composition of the invention and the liquid analyte by shaking thereof, a vessel that has a cylindrical shape or the like and is of a material that is hard to cause deformation is preferred. Moreover, for example, in order to mix the composition of the invention and the liquid analyte by rubbing and pressing together with the vessel, a vessel of a material that is easily deformed and flexible is preferred. Specific examples of the vessel preferably include a bag-shaped vessel of a polymer such as a polyvinyl base and a polyethylene base, and a vessel with a sealing device such as a lid and a fastener is further preferred. Moreover, the culture vessel is preferably transparent in view of easiness of counting the colonies of the microorganism from outside of the vessel. A dose capable of being housed therein is not particularly limited, but specific examples thereof preferably include 100 milliliters to 1,000 milliliters, which is suitable for applying the vessel to a large volume of analyte containing a small amount of microorganisms.

While the analyte has been so far brought into contact with the culture equipment containing the preformed medium to culture and count the microorganisms, the culture equipment for counting the number of microorganisms according to the invention is different therefrom in that the equipment is preferable for a use aspect in which the polymer compound is gelled by using the liquid analyte itself as a solvent in the culture vessel, and the microorganisms in the analyte are cultured inside the gel and the resulting material is provided for counting the microorganisms.

Moreover, the culture vessel may be formed into a plate (sheet) form, and such a culture vessel is suitable for detecting the microorganisms by using a small amount of analyte, for example about 1 milliliter of the analyte.

Specific examples thereof include a form of a housing vessel prepared by stacking a general petri dish or a concave dish-shaped sheet and a plate-shaped or convex sheet. The colonies of the microorganisms can be further easily counted by forming the vessel into the plate-shaped form. Moreover, use of the small-size vessel facilitates processing of a plurality of analytes in parallel at one time.

In the case of such a small size and a plate-shape culture vessel, the culture vessel can also be used for a diluted analyte, and therefore such a vessel becomes preferable also when the number of the microorganisms in the analyte is 300 CFU/mL or less, for example.

The composition for preparing the medium for counting the number of microorganisms according to the invention as described above can be preferably used in the counting method of the invention.

The counting method of the invention includes a step of adding the analyte to the composition of the invention and mixing the resulting mixture, a step of culturing the microorganisms contained in the analyte, and a step of counting the number of colonies of the microorganisms.

Mixing of the composition of the invention with the liquid analyte can be performed by an arbitrary method, and for example, the mixing may be performed by shaking or rubbing both together with the vessel or by stiffing with a sterile device.

Culturing conditions of the microorganisms are not particularly limited, and are properly selected according to a kind of the target microorganisms, but are preferably 24 to 48 hours at 35±2° C., for example.

The colonies formed by growth of the target microorganisms emerge in the medium after culture, and the colonies can be confirmed by visual observation, and bubbles of carbon dioxide gas is hard to be generated, and therefore the number of the colonies can be accurately counted.

The counting method of the invention can be preferably used for an analyte in which an amount of existing microorganisms is small, more specifically cleanliness is high. For example, the counting method is preferred when the number of microorganisms in the analyte is 0.1 CFU/mL or less at a level at which the microorganisms are unable to be detected by ordinary inspection using 1 milliliter.

In general, if the analyte in which the amount of existing microorganisms is large, the colonies can be counted by appropriately diluting the analyte so as to be suitable for the detection method, but when the amount of existing microorganisms is small, concentration is complicated or difficult in several cases. Even in such a case, the counting method of the invention is useful in that the number of microorganisms can be simply and accurately detected.

Moreover, the counting method of the invention is also useful even for a large amount of the liquid analyte in view of capability of directly providing the analyte for counting without applying pretreatment thereto. For example, the counting method is preferred when the analyte weight is high in corresponding to a hydration ability of (a) sodium polyacrylate in the composition according to the invention, for example 10 to 10,000 times the weight of the analyte. Alternatively, the counting method is preferred when the analyte is in a large amount, for example, in 100 milliliters or more.

The analyte to which the counting method according to the invention can be applied is not particularly limited, and specific examples thereof ordinarily include a liquid analyte containing carbonate ion or bicarbonate ion in an amount of 0.005 mol/100 mL or more. Specific examples of such a liquid analyte preferably include dialysis water. Moreover, the analyte may be a culture solution obtained by preculturing such an analyte in tryptic soy broth or the like.

### EXAMPLES

Next, the invention will be described in greater detail by way of Examples. The invention is not limited by the Examples.

### Reference Example 1

(1) Preparation of Composition for Preparing Medium for Counting Microorganisms

A composition was prepared by mixing a material having a formulation shown in Table 1 in a transparent and colorless plastic vessel having a cylindrical shape and a volume of 150 mL.

TABLE 1

| Material name | Composition (g) in 100 mL |
|---|---|
| Yeast extract | 0.05 |
| Peptone | 0.05 |
| Casamino acid | 0.05 |
| Glucose | 0.05 |
| Soluble starch | 0.05 |
| Sodium pyruvate | 0.03 |
| Potassium hydrogenphosphate | 0.03 |
| Magnesium sulfate heptahydrate | 0.005 |
| TTC | 0.0025 |
| Sodium polyacrylate | 1 |
| | pH 7.2 |

* As sodium polyacrylate, Aqualic CA (Nippon Shokubai Co., Ltd.) was used.

(2) Preparation of Strain Inoculated

As a specimen strain, Bacillus subtilis NBRC3134 was used. The specimen strain was pre-cultured in a tryptic soy agar medium for 24 hours, and then was suspended into sterile physiological saline using a sterile swab to be a concentration corresponding to McFarland Turbidity Standard No. 1 (about 3.0×10⁸ CFU/mL), and taken as a bacteria stock solution. Then, a bacteria diluted solution having a concentration of several CFU/mL was prepared by repeating 10-fold step dilution of the bacteria stock solution with the sterile physiological saline into a concentration of 10⁸ CFU/mL. Then, 1 mL of the bacteria diluted solution was added to 99 mL of sterile water, and the resulting material was taken as a sample solution in which only a significantly small amount of microorganisms, namely only several CFU/100 mL thereof, existed. Then, 100 mL of the sample solution was added to the composition prepared in (1) described above, and the resulting material was vertically shaken and mixed for several minutes to solidify the material at room temperature. After the solidified medium was allowed to stand, the resulting material was cultured at 35° C. for 24 hours, and then presence or absence of growth was confirmed.

(3) Results

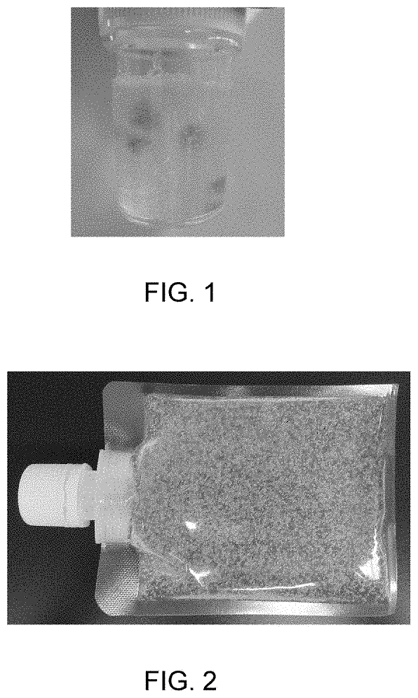

FIG. 1 shows colonies of Bacillus subtilis.

The sample solution of the specimen strain was quickly solidified by using the composition in Reference Example 1, and after culture, as shown in FIG. 1, red colonies were able to be confirmed in transparent gel, and the number thereof was able to be easily counted.

### Example 1 and Comparative Example 1

(1) Preparation of Composition for Preparing Medium for Counting Microorganisms

A material having a formulation shown in Table 2 was mixed in a transparent and colorless plastic spout bag having a volume of 100 mL to prepare a composition in Comparative Example 1.

Moreover, 0.3 g/100 mL of calcium hydroxide was also added to the material having the formulation shown in Table 2, and then the resulting material was mixed in a transparent and colorless plastic spout bag having a volume of 100 mL to prepare a composition in Example 1. In addition, a concentration of calcium hydroxide in the composition in Example 1 when in use corresponds to 25 mol % in a sodium hydrogencarbonate content in 100 mL of a dialysis fluid LYMPACK TA3 (made by Nipro Corporation) to be added in the section (2) described below.

TABLE 2

| Material name | Composition (g) in 100 mL |
|---|---|
| Casein peptone | 0.25 |
| Yeast extract | 0.125 |
| Glucose | 0.05 |
| Sodium polyacrylate | 2 |
| Sodium carbonate | 0.2 |
| TTC | 0.002 |
| | pH 7.2 |

* As sodium polyacrylate, Aqualic CA (Nippon Shokubai Co., Ltd.) was used.

(2) Preparation of Dialysis Fluid

A dialysis fluid prepared using LYMPACK TA3 (made by Nipro Corporation) according to an attachment was added by 100 mL to each vessel in which a composition in Example 1 or Comparative Example 1 was housed, and the resulting material was sufficiently mixed by rubbing the vessel for several minutes to solidify the resulting material at room temperature. Then, the vessel was allowed to stand at 35° C. for 24 hours, and then an aspect of the medium was observed.

(3) Results

As shown in FIG. 2, in the medium prepared using the composition in Comparative Example 1, uncountable bubbles presumable as a carbon dioxide gas were generated, and the medium appeared white cloudy.

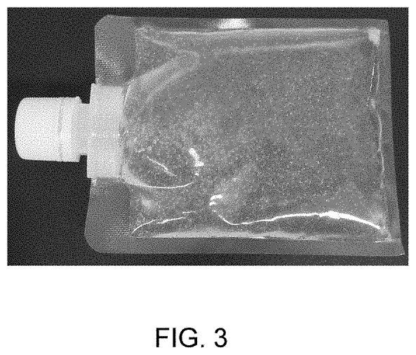

In contrast, as shown in FIG. 3, in the medium prepared using the composition in Example 1, generation of bubbles presumable as a carbon dioxide gas was suppressed, and visibility upon counting microorganisms was improved. In addition, even when the composition in Example 1 was used, a gelation speed was not different from the speed in Comparative Example 1.

### INDUSTRIAL APPLICABILITY

According to the invention, even in an analyte containing carbonate ion or bicarbonate ion, the number of microorganisms in the analyte can be simply and accurately counted. In particular, quantitative detection can be achieved even for a small amount of microorganism counts existing in a large volume of analyte, and therefore also with regard to an analyte in which cleanliness is high, the number of a trace amount of microorganism counts existing therein can be thoroughly counted with high visibility.

1. Use of a composition for counting the number of microorganisms, wherein the composition comprises (a) polyacrylic acid and/or a salt thereof, (b) hydroxide of alkaline earth metal and (c) a nutritional ingredient.

2. The Use of claim 1, wherein the composition further comprises (d) a coloration reagent.

3. The Use of claim 1, wherein (b) the hydroxide of alkaline earth metal is selected from the group of calcium hydroxide, magnesium hydroxide, barium hydroxide, strontium hydroxide, radium hydroxide and beryllium hydroxide.

4. Use of culture equipment for counting the number of microorganisms, wherein the culture equipment comprises (a) polyacrylic acid and/or a salt thereof, (b) hydroxide of alkaline earth metal, (c) a nutritional ingredient, and a culture vessel.

5. A composition for preparing a medium for counting the number of microorganisms, comprising (a) polyacrylic acid and/or a salt thereof, (b) hydroxide of alkaline earth metal and (c) a nutritional ingredient.

6. The composition according to claim 5, further comprising (d) a coloration reagent.

7. The composition according to claim 5, wherein (b) the hydroxide of alkaline earth metal is selected from the group of calcium hydroxide, magnesium hydroxide, barium hydroxide, strontium hydroxide, radium hydroxide and beryllium hydroxide.

8. Culture equipment for counting the number of microorganisms, comprising the composition according to claim 5, and a culture vessel.

9. A counting method for the number of microorganisms, comprising:
a step of adding an analyte to the composition according to claim 5 and mixing the resulting mixture;
a step of culturing microorganisms contained in the analyte; and
a step of counting the number of colonies of the microorganisms.

10. The counting method according to claim 9, wherein the analyte contains 0.005 mol/100 mL or more of carbonate ion or bicarbonate ion.

11. The counting method according to claim 9, wherein the number of microorganisms in the analyte is 0.1 CFU/mL or less.

12. The counting method according to claim 9, wherein a weight of the analyte is 10 to 10,000 times a weight of (a) the polyacrylic acid and/or the salt thereof in the composition.
